(19) United States
(12) Patent Application Publication
(10) Pub. No.: US 2026/0096905 A1
(43) Pub. Date: Apr. 9, 2026

(54) IMPLANTABLE DRUG-DEVICE COMBINATIONS, AND RELATED METHODS OF TREATMENT

(71) Applicant: PITUVIA THERAPEUTICS, INC., San Diego, CA (US)

(72) Inventor: Patrick J. Fitzgerald, San Diego, CA (US)

(21) Appl. No.: 19/352,375

(22) Filed: Oct. 7, 2025

Related U.S. Application Data
(60) Provisional application No. 63/704,368, filed on Oct. 7, 2024.
(60) Provisional application No. 63/796,915, filed on Apr. 29, 2025.

(51) Int. Cl. A61F 2/46; A61F 2/30; A61L 27/18; A61L 27/22

(52) U.S. Cl. CPC A61F 2/46; A61L 27/18; A61L 27/227; A61F 2002/30062; A61F 2002/30677; A61L 2300/43; A61L 2300/432; A61L 2300/622; A61L 2300/624

(57) ABSTRACT

Provided are drug-device combinations and methods used for direct delivery of therapeutic drugs to the pituitary gland, wherein the drug-device combination also acts as a barrier between a sella turcica (pituitary fossa) and a sphenoid sinus to effectively trap or confine the drug substance within the sella turcica to prevent its premature escape away into the CSF from the target pituitary; thereby reducing or eliminating systemic toxicity of the drug substance. The drug-device combination includes a cap, a stem, a drug that is therapeutically effective for a pituitary gland disorder.

### TECHNICAL FIELD

The present invention relates generally to an implantable drug-device combination used for direct delivery of therapeutic drugs to the pituitary gland, and related methods of treatment.

### BACKGROUND

During transphenoidal surgeries such as endoscopic transphenoidal pituitary tumor removal, a surgeon typically re-creates the barrier between the sphenoid sinus and sella turcica by using an inlay, an overlay, or a combination of the two to re-create the barrier.

Inlay materials are placed on the brain side of the sella turcica. Inlay materials serve as structural support to counter the ongoing downward forces of the spinal fluid and meninges resulting from spinal fluid pressure and gravity. Inlay materials include bone, cartilage, and other less structurally sound materials such as fascia, pericardium, or dura harvested from the patient, or provided from a tissue bank. Bone and cartilage may provide significant support to prevent the slow, persistent herniation of meninges in the days and weeks after surgery leading to cerebrospinal fluid leaks. However, bone is difficult to work with due to sharp edges that may need to be removed, and due to the small custom shape needed to be created. The bone needs to be sized larger than the hole, making it difficult to place through the craniotomy. Patient cartilage is often too weak, and cracks when handling. While cadaveric rib cartilage is nearly ideal, it is a bit too rigid and time consuming to customize. Cadaveric rib cartilage is also not readily available, and is expensive.

Overlay materials do not provide long lasting structural support. Some overlays “plug” the hole using fat grafts, cellulose packing materials, or injectable glues. Other overlays cover the hole with fascia, dura, or mucosal grafts. Overlay materials such as mucosa, fat, gel foam, surgical glue, collagen, etc. are easy to place and readily available. Fat requires a separate abdominal incision. These materials provide no structural support and rely on the hope that tissue ingrowth and healing will occur before the herniation of meninges and cerebrospinal fluid overcome them. Overlays are often sufficient for small meningeal penetrations; however, for medium to larger cerebrospinal fluid leaks, overlays are generally insufficient.

Overlays often lead to delayed cerebrospinal fluid leaks. The tissue used in the overlays often resorbs or mobilizes out of position with development of a cerebrospinal fluid leak.

U.S. Patent Application No. 2007/0270841 to B. Badle discloses implantable devices that may be positioned, for example, into the sphenoid sinus or sella turcica of a subject. In certain embodiments, the implantable devices comprise a center plate and a plurality of protruding arms extending from the center plate, both of which may be composed of bioresorbable material.

Although current drugs used for pituitary gland related diseases are effective, they are known to have considerable systemic toxicity. Thus, there is a need to for new devices and methods to reduce the considerable systemic toxicity of current therapies directed at the pituitary gland.

### SUMMARY

Provided herein are drug-device combinations and methods used for direct delivery of therapeutic drugs to the pituitary gland, wherein the drug-device combination also acts as a barrier between a sella turcica (pituitary fossa) and a sphenoid sinus to effectively trap or confine the drug substance within the sella turcica to prevent its premature escape away into the CSF from the target pituitary; thereby reducing or eliminating systemic toxicity of the drug substance.

In one embodiment, the drug-device combination includes a cap and a stem. The cap is formed of a deformable material. The cap includes a contact surface and an upper surface. The contact surface forms a base of the cap and includes an outer perimeter. The upper surface includes an apex offset from the contact surface. The upper surface extends from the apex to the outer perimeter. The stem extends from the contact surface in a direction opposite the apex.

A further embodiment includes a method for placing a drug-device combination in an inlay position in the sella turcica. The drug-device combination includes a cap formed of deformable material and a stem extending from the cap. The method includes gripping the drug-device combination using a surgical tool. The method also includes introducing the drug-device combination into the sphenoid sinus. The method further includes passing the drug-device combination through a hole in the sphenoid bone by deforming the cap. The method still further includes locating the drug-device combination with its original shape such that the cap is inside the sella turcica and a contact surface of the cap is in contact with the sphenoid bone about the hole.

Also provided herein, are drug-device combinations and methods used for direct delivery of therapeutic drugs to the pituitary gland, wherein the drug-device combination is a drug-eluting implant and/or microparticle (e.g., microsphere, nanoparticle, and the like) inserted into the sella turcica to effectively deliver the drug within an enclosed sella. Drug or drug-eluting compositions comprising microimplants (FIG. 15-19) and/or microparticles (FIG. 14), such as microspheres (beads), elongated microparticles, and the like, are advanced via a catheter into the sella turcica. The drug-eluting compositions can be bio-durable or bio-absorbable (e.g., bio-degradable), as set forth herein. Once the drug or drug-eluting composition is administered into the sella turcica, the catheter is removed and the surgical site is closed.

An advantage of this aspect is that there will be no implant left within the sphenoid sinus, thereby reducing bioincompatibility issues. Another advantage of this aspect, is that the area can be reaccessed in a similar fashion to apply additional drug or drug-eluting implants or microparticles, e.g., microspheres or nanospheres, and the like.

Accordingly, also provided herein are improved methods of treating pituitary gland-related diseases comprising contacting the pituitry gland with a drug substance that has eluted from the invention drug-device combinations provide herein. In certain embodiments, provided herein are methods of treating a disease by delivering a drug substance directly to a pituitary gland, said method comprising: forming a hole in a sphenoid bone that acts as a barrier between a sella turcica and a sphenoid sinus; and inserting the invention drug-device combination provided herein. In certain embodiments, the drug substance is selected from somatostatin receptor ligands (SRL), dopamine agonists, Gonadotropin Releasing Hormone (GnRH) agonists and/or GnRH antagonists. In particular embodiments, the drug substance is selected from the group consisting of: pasireotide, octreotide, lanreotide, paltusotine, cabergoline, leuprolide, goserelein, triptorelin, cetrorelix, elagolix, and/or relugolix. In particular embodiments, the disease is selected from: acromegaly, Cushing's disease, thyrotrophinomas, diarrhea, gastroenteropancreatic neuroendocrine tumors (GEP-NETs), carcinoid syndrome, hyperprolactinemia, Parkinsonian Syndrome, prostate cancer and/or breast cancer.

Accordingly, provided herein are the following items:
1. A drug-device combination for direct delivery of a drug to a pituitary gland, the drug-device comprising:
   a cap formed of a deformable material, the cap including
      a contact surface forming a base of the cap, the contact surface including an outer perimeter, and
      an upper surface including an apex offset from the contact surface at a cap height, the upper surface extending from the apex to the outer perimeter;
   a stem extending from the contact surface in a direction opposite the apex, wherein the drug-device combination covers a hole formed in a sphenoid bone between a sella turcica and a sphenoid sinus; and
   a drug substance that is therapeutically effective for a pituitary gland disorder.
2. The drug-device combination of item 1, wherein the drug substance is selected from a somatostatin receptor ligand (SRL) or a dopamine agonist.
3. The drug-device combination of items 1-2, wherein the drug substance is selected from the group consisting of: pasireotide, octreotide, lanreotide, paltusotine, cabergoline, leuprolide, goserelein, triptorelin, cetrorelix, elagolix, and/or relugolix.
4. The drug-device combination of items 1-3, wherein the deformable material is an elastic material.
5. The drug-device combination of items 1-4, wherein the deformable material includes a hardness up to Shore 20 A.
6. The drug-device combination of items 1-5, wherein the deformable material is ethylene vinyl acetate (EVA).
7. The drug-device combination of items 1-6, wherein the upper surface extends from the apex to the outer perimeter in a curved manner.
8. The drug-device combination of items 1-7, wherein the upper surface includes a spherical cap shape.
9. The drug-device combination of items 1-8, wherein the contact surface is an annular surface and the stem includes a cylindrical surface extending from an inner diameter of the contact surface.
10. The drug-device combination of items 1-9, wherein the contact surface and the stem are aligned on a single axis.
11. The drug-device combination of items 1-10, wherein the stem includes a body portion with a first cylindrical shape including a first diameter and a neck portion extending between the cap and the body portion with a second cylindrical shape, the second cylindrical shape including a second diameter smaller than the first diameter.
12. The drug-device combination of items 1-11, further comprising a gripping feature located at an end of the stem distal to the cap.
13. The drug-device combination of item 12, wherein the gripping feature includes a ridge.
14. The drug-device combination of item 13, wherein the ridge is formed by adjacent recesses in the end of the stem distal to the cap.
15. The drug-device combination of items 1-14, wherein a radius of the upper surface is larger than the cap height.
16.The drug-device combination of items 1-15, wherein the cap is a frustum and includes a tip surface at the apex, the tip surface being parallel to the contact surface and spaced apart from the contact surface at the cap height.
17. The drug-device combination of items 1-16, wherein the cap is an ellipsoid and the contact surface is an elliptical surface where the cap width is a major axis from 10 millimeters to 30millimeters and the contact surface also includes a minor axis from 6 millimeters to 20 millimeters.
18. The drug-device combination of items 1-17, wherein the cap include multiple legs converging together forming the upper surface, each leg including a flat surface that forms a portion of the contact surface.
19. The drug-device combination of items 1-18, wherein the stem is removable.
20. The drug-device combination of items 1-19, further comprising a second material that acts as a marker for location of the drug-device combination through imaging.
21. A drug-device combination for closing a hole in a sphenoid bone that acts as a barrier between a sella turcica and a sphenoid sinus, the drug-device combination comprising:
   a cap with a spherical cap shape, the cap including
      an upper surface with a spherical shape, and
      a contact surface forming a base of the spherical cap shape, the contact surface being an annular surface;
   a stem extending from the contact surface in a direction opposite the upper surface; and
   a drug substance that is therapeutically effective for a pituitary gland disorder.
22. The drug-device combination of item 21, wherein the drug substance is selected from a somatostatin receptor ligand (SRL) or a dopamine agonist.
23. The drug-device combination of items 21-22, wherein the drug substance is selected from the group consisting of: pasireotide, octreotide, lanreotide, paltusotine, cabergoline, leuprolide, goserelein, triptorelin, cetrorelix, elagolix, and/or relugolix.
24. The drug-device combination of items 21-23, wherein the stem includes a body portion with a first cylindrical shape including a first diameter, and a neck portion extending between the cap and the body portion with a second cylindrical shape, the second cylindrical shape including a second diameter smaller than the first diameter.
25. The drug-device combination of items 21-24, wherein the upper surface and the contact surface intersect at an outer perimeter.
26. The drug-device combination of items 21-25, wherein the cap includes a cap width from 6millimeters to 40 millimeters, the cap width being a diameter of the cap at an intersection of the upper surface and the contact surface.
27. The drug-device combination of items 21-26, wherein the stem includes a stem width from 4 millimeters to 7 millimeters.
28. The drug-device combination of items 21-27, wherein the stem extends from the cap from 5 millimeters to 10 millimeters.
29. The drug-device combination of items 21-28, further comprising a gripping feature located at an end of the stem distal to the cap, the gripping feature including slots forming a ridge there between.
30. The drug-device combination of items 21-29, wherein the cap is formed from a material with a hardness up to Shore 20 A.
31. The drug-device combination of items 21-30, wherein the drug-device combination is configured to elute drug substance into the sella turcica for direct contact with the pituitary gland, wherein the drug substance is effectively confined within the sella turcica and prevented from contact with the CSF or blood system.
32. The drug-device combination of items 21-31, wherein the drug-device combination is configured to allow for intracranial pressure relief.
33. The drug-device combination of items 21-32, wherein the device is formed from a material corresponding to ethylene vinyl acetate (EVA).
34. A method for locating a drug-device combination in an inlay position in the sella turcica, the drug-device combination including a cap formed of deformable material and a stem extending from the cap, the method comprising:
   gripping the drug-device combination of item 1 using a surgical tool;
   introducing the drug-device combination into the sphenoid sinus;
   passing the drug-device combination through a hole in the sphenoid bone by deforming the cap; and
   locating the drug-device combination with its original shape such that the cap is inside the sella turcica and a contact surface of the cap is in contact with the sphenoid bone about the hole.
35. The method of item 34, wherein gripping the drug-device combination using a surgical tool includes gripping a ridge of a gripping feature located at an end of the stem distal to the cap.
36. A method of treating a disease by delivering a drug substance directly to a pituitary gland, said method comprising: forming a hole in a sphenoid bone that acts as a barrier between a sella turcica and a sphenoid sinus; and inserting a drug-device combination implant.
37. The method of item 36, wherein the drug-device combination implant is inserted by using a catheter, needle or gripping the drug-device combination using a surgical tool; introducing the drug-device combination implant into the sphenoid sinus; passing the drug-device combination implant through a hole in the sphenoid bone by deforming the drug-device combination implant; and locating the drug-device combination implant such that the drug-device combination implant is inside the sella turcica and in contact with the sphenoid bone about the hole.
38. The method of items 36-37, wherein the drug-device combination implant regains its original shape.
39. The method of items 36-38, wherein the drug substance is selected from a somatostatin receptor ligand (SRL) or a dopamine agonist.
40. The method of items 36-29, wherein the drug substance is selected from the group consisting of: pasireotide, octreotide, lanreotide, paltusotine, cabergoline, leuprolide, goserelein, triptorelin, cetrorelix, elagolix, and/or relugolix.
41. The method of items 36-40, wherein the disease is selected from: acromegaly, Cushing's disease, thyrotrophinomas, diarrhea, gastroenteropancreatic neuroendocrine tumors (GEP-NETs), carcinoid syndrome, hyperprolactinemia, Parkinsonian Syndrome, prostate cancer and/or breast cancer.
42. The method of items 36-41, wherein the drug-device combination implant is configured to elute drug substance into the sella turcica for direct contact with the pituitary gland, wherein the drug substance is effectively confined within the sella turcica and prevented from contact with the CSF or blood system.
43. A method for placing a drug-device combination within a pituitary fossa, the method comprising:
   forming a hole in a sphenoid bone that acts as a barrier between a sella turcica and a sphenoid sinus using a surgical tool;
   introducing the drug-device combination into the pituitary fossa via an introducer inserted into the hole;
   withdrawing the introducer; and
   surgically closing the hole,
   wherein the drug-device combination comprises a drug substance and an implant and/or microparticle.
44. The method of item 43, wherein the introducer is a needle or catheter.
45. The method of item 43-44, wherein the drug substance is selected from somatostatin receptor ligands (SRL), dopamine agonists, Gonadotropin Releasing Hormone (GnRH) agonists and/or GnRH antagonists.
46. The method of item 43-45, wherein the drug substance is selected from the group consisting of: pasireotide, octreotide, lanreotide, paltusotine, cabergoline, leuprolide, goserelein, triptorelin, cetrorelix, elagolix, and/or relugolix.
47. The method of items 43-46, wherein the drug-device combination implant is configured to elute drug substance into the sella turcica for direct contact with the pituitary gland, wherein the drug substance is effectively confined within the sella turcica and prevented from contact with the CSF or blood system.
48. A drug-device combination for direct delivery of a drug to a pituitary gland, the drug-device comprising:
   an implant formed of a deformable material; and
   a drug substance that is therapeutically effective for a pituitary gland disorder.
49. The drug-device combination of item 48, wherein the drug substance is selected from a somatostatin receptor ligand (SRL) or a dopamine agonist.
50. The drug-device combination of items 48-49, wherein the drug substance is selected from the group consisting of: pasireotide, octreotide, lanreotide, paltusotine, cabergoline, leuprolide, goserelein, triptorelin, cetrorelix, elagolix, and/or relugolix.
51. The drug-device combination of items 48-50, wherein the deformable material is an elastic material.
52. The drug-device combination of items 48-51, wherein the deformable material is ethylene vinyl acetate (EVA).
53. The drug-device combination of items 48-52, wherein the implant comprises a microparticle, microspheres and/or nanosphere.

### BRIEF DESCRIPTION OF THE DRAWINGS

FIG. 1 is a side view of an exemplary embodiment of a cranial base implant device.

FIG. 2 is a bottom view of the cranial base implant drug-device combination of FIG. 1.

FIG. 3 is a cross-sectional view of an alternate embodiment of the cranial base implant drug-device combination of FIG. 1.

FIG. 4 is a bottom view of an alternate embodiment of the cranial base implant drug-device combination of FIG. 1.

FIG. 5 is a bottom view of an alternate embodiment of the cranial base implant drug-device combination of FIG. 1.

FIGS. 6-8 illustrate the cranial base implant drug-device combination of FIG. 1 passing through a hole in the sphenoid bone.

FIGS. 9-12 illustrate the insertion of the cranial base implant drug-device combination into the sella turcica.

FIG. 13 is a flowchart of a method for placing (or locating) the drug-device combination 100 in an inlay position in the sella turcica 210.

FIG. 14 illustrates the insertion of the drug-eluting drug-device combination microparticles within the sella turcica.

FIG. 15 illustrates the insertion of the drug-eluting drug-device combination implant (microimplant) within the sella turcica.

FIG. 16 illustrates an exemplary drug-device clover implant (e.g., a microimplant) having flanges A-D.

FIG. 17 illustrates a frontal view of the drug-device clover implant 1710 loaded in a catheter 1720.

FIG. 18 illustrates a side view of drug-device clover implant 1810 loaded in a catheter 1820, whereby the implant is advanced by the inner introducer advancer 1830. The “D” flange from FIGS. 16 and 17 is hidden from view. In this embodiment, the implant 1810 has nodules 1840 on its leading edge for stability.

FIG. 19 illustrates an exemplary drug-device clover implant position within the sella turcica after use of the invention methods and drug-devices.

### DETAILED DESCRIPTION

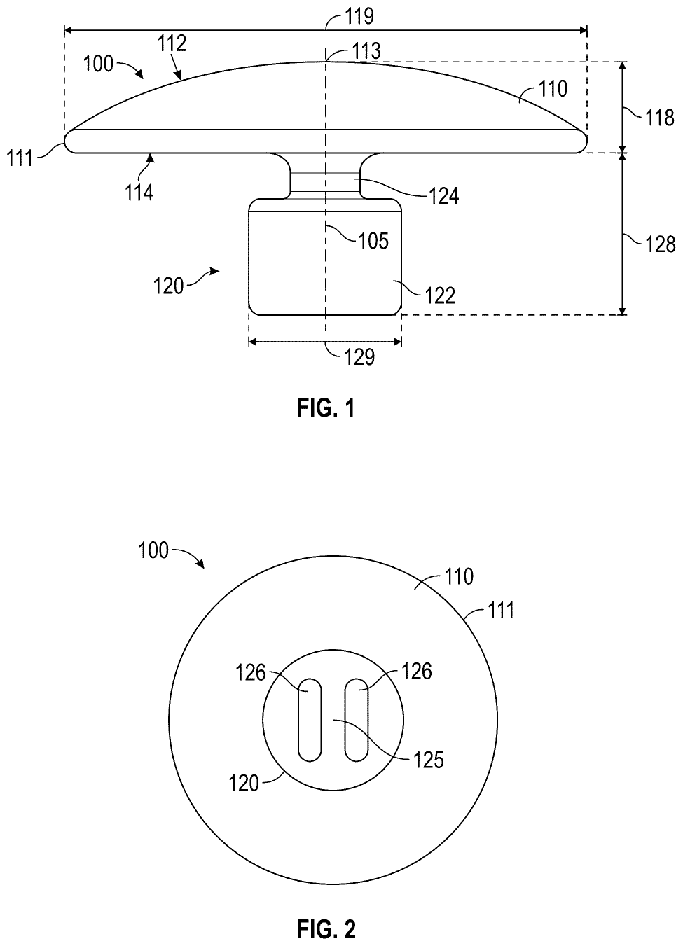

Provided herein are drug-device combinations and methods used for direct delivery of therapeutic drugs to the pituitary gland, wherein in one embodiment, the drug-device combination also acts as a barrier between a sella turcica and a sphenoid sinus to effectively trap the drug within the sella and prevent its premature escape away from the target, the pituitary. In this embodiment set forth in FIGS. 1-13, the drug-device combination disclosed herein includes a mushroom or conical shaped compressible or deformable biocompatible material soft enough to be atraumatic to bone and surrounding neurovascular structures, yet rigid enough to hold form and provide support after insertion into the sella turcica through a hole smaller than an outer diameter of the contact surface or the maximum width of the device.

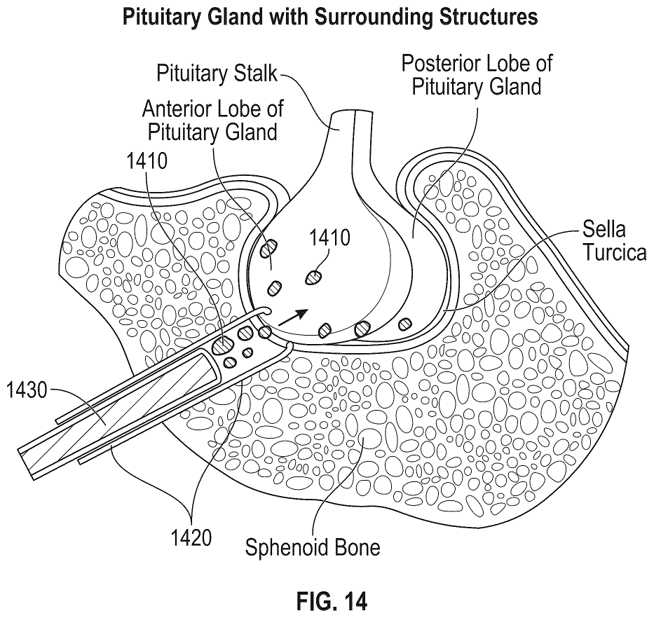

In another embodiment set forth in FIGS. 14-17, the drug-device combination is a drug-eluting implant or microparticle (e.g., microsphere, nanoparticle, and the like) inserted into the pituitary fossa to effectively deliver the drug within an enclosed sella turcica.

As used herein, the phrase “drug-device combination” refers to a structure that comprises a therapeutic agent (drug) in a format that can be delivered or eluted into its surroundings over time. Exemplary structural formats of the drug-device combinations include the implants and microparticles described herein, and the like.

FIG. 1 is a side view of an exemplary embodiment of a cranial base implant drug-device combination (“device”) 100. drug-device combination 100 includes a cap 110 and a stem 120. Cap 110 may be rounded and may have a spherical, ellipsoidal or conical shape. The ellipsoidal shapes may include oblate and prolate spheroids. In the embodiment shown in FIG. 1, cap 110 is the shape of a spherical cap, a region of a sphere above a given plane; more particularly cap 110 is a spherical cap with a cap height 118 less than the radius of the sphere. In some embodiments, cap 110 is the shape of a hemisphere, a spherical cap with the plane cutting through the center of the sphere. In other embodiments, cap 110 is a spherical cap with a cap height 118 substantially smaller than the radius of the sphere resulting in a plate like cap 110 with a slightly domed upper surface 112. The cap 110 includes a cap width 119, which is the largest width of cap 110, which may be at the base of the spherical cap and may be the diameter of the base of the spherical cap. In one embodiment, cap width 119 is from 6 millimeters to 20 millimeters. In another embodiment, cap width 119 is from 20 millimeters and 40 millimeters. In yet another embodiment, cap width 119 is from 6 millimeters to 40 millimeters. In still another embodiment, cap width 119 is up to 40 millimeters.

Cap 110 includes a contact surface 114 and an upper surface 112. Contact surface 114 includes an outer perimeter 111. All or a portion of the outer perimeter 111 may be defined by cap width 119. In some embodiments, outer perimeter 111 is a circular shape as illustrated in FIG. 2. Contact surface 114 may have an annular shape, such as an annular surface. The outer diameter of the annular shape may match the outer perimeter 111. The inside diameter of the annular shape may match a diameter of stem 120.

Upper surface 112 includes an apex 113 spaced apart from the contact surface 114 at a cap height 118. The apex 113 may be a point or a surface. Upper surface 112 may extend from the apex 113 to the outer perimeter 111. In some embodiments, upper surface 112 extends from the apex 113 to the outer perimeter 111 in a curved manner. Upper surface 112 may have spherical, ellipsoidal or conical surface shape.

Cap 110 may be a spherical, ellipsoidal, spheroidal, or conical cap or frustum. A spherical, ellipsoidal, spheroidal, or conical frustum is the solid defined by cutting a sphere, ellipsoid, spheroid, or cone with a pair of parallel planes.

Stem 120 extends from cap 110. Stem 120 may extend from cap 110 at a stem length 128 and may extend in the direction opposite apex 113. Stem length 128 may be from 5 millimeters to 10 millimeters. Stem 120 may generally include a cylindrical shape and may include a stem width 129. The stem width 129 may be the largest width of the stem 120, such as the outer diameter of the cylindrical shape. Stem width 129 is generally smaller than cap width 119. Stem width 129 may be from 4 millimeters to 7 millimeters.

In a particular embodiment, the cap includes a cap width 119 from 6 millimeters, the cap width being a diameter of the cap at an intersection of the upper surface and the contact surface; the stem width 129 is 4 millimeters; and the stem extends from the cap corresponding to a stem length 128 of 5 millimeters.

Stem 120 may include a body portion 122 and a neck portion 124. Body portion 122 may include a cylindrical shape with a diameter matching stem width 129. Neck portion 124 may extend between body portion 122 and contact surface 114. Neck portion 124 may include a diameter smaller than body portion 122 and may be smaller than stem width 129. The symmetry of upper surface 112, contact surface 114, body portion 122, and neck portion 124 may be about a single axis 105.

The diameter of neck portion 124 may match the inside diameter of contact surface 114. In the embodiment shown in FIG. 1, stem 120 extends from contact surface 114 in a direction perpendicular to contact surface 114.

In some embodiments, the cap 110 and the stem 120 may be symmetrical about the axis 105. The contact surface 114 and the stem 120 may also be aligned on axis 105. In other embodiments, the cap 110 and the stem 120 may be asymmetric. The various edges of the drug-device combination 100 may be rounded. Stem 120 may be an integral piece to cap 110 or may be removable from cap 110.

FIG. 2 is a bottom view of the drug-device combination 100 of FIG. 1. As illustrated in FIG. 2, drug-device combination 100 may include a gripping feature 126. Gripping feature 126 may be located at the end of stem 120 distal to cap 110. In the embodiment illustrated in FIG. 2, gripping feature 126 includes adjacent recesses, such as slots extending into the end of stem 120 distal to cap 110 forming a ridge 125 there between. In other embodiments, ridge 125 may protrude from the end of stem 120 distal to cap 110. Gripping feature 126, and in particular ridge 125, may be configured for the interlocking of instruments such as Blakesly forceps and the like.

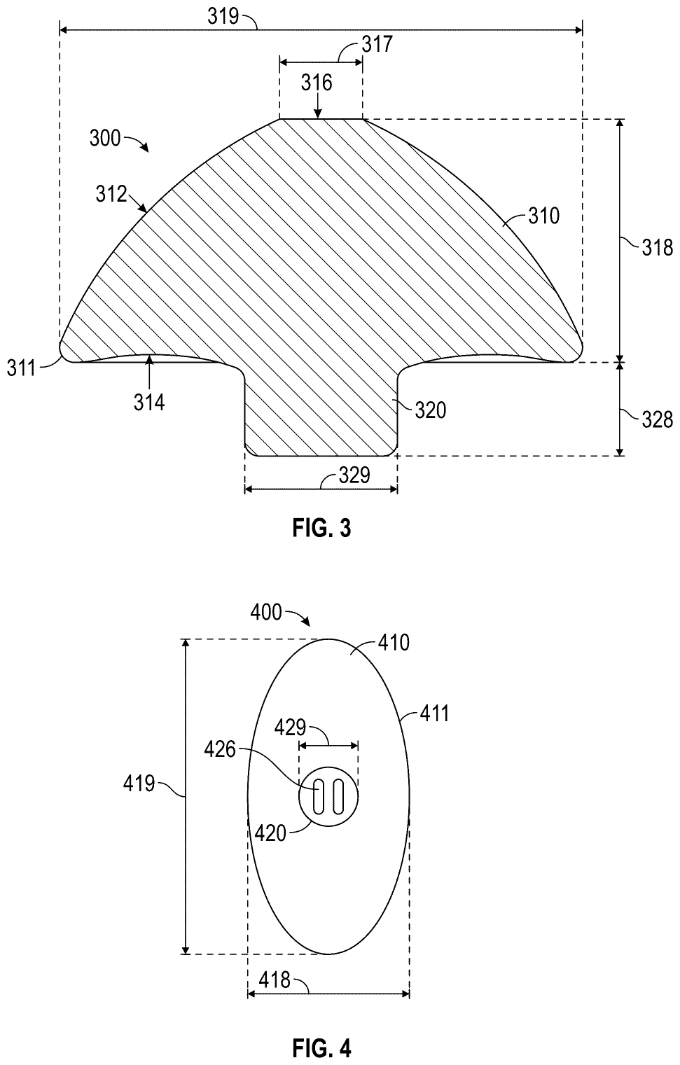

FIG. 3 is a cross-sectional view of an alternate embodiment of the drug-device combination 100 of FIG. 1. In the embodiment shown in FIG. 3, cap 310 of drug-device combination 300 is rounded and includes a prolate spheroid frustum where the parallel planes cut through the major axis of the prolate spheroid on the same half of the prolate spheroid. In one embodiment, one parallel plane cuts through the prolate spheroid to define contact surface 314 and the other plane cuts through the prolate spheroid near or at the vertex of the prolate spheroid, forming tip surface 316. Tip surface 316 may be the apex of upper surface 312. The tip width 317 of tip surface 316 is smaller than the cap width 319.

In some embodiments, such as the embodiment illustrated in FIG. 3, contact surface 314 is an annular surface with a curved profile forming a concave surface that is revolved about stem 120. The various components and dimensions of drug-device combination 300, such as cap width 319, cap height 318, stem width 329, and stem height 328 of drug-device combination 300 may be the same or similar to the cap width 119, cap height 118, stem width 129, and stem height 128 as described in conjunction with drug-device combination 100.

FIG. 4 is a bottom view of an alternate embodiment of the devices 100 of FIGS. 1 and 300 of FIG. 3. In the embodiment illustrated, cap 410 includes an elliptical contact surface 414. Cap 410 may be an ellipsoidal cap or frustum, where the cutting surface(s) is parallel to the major axis of the ellipsoid, forming the elliptical outer perimeter 411 of contact surface 414. Cap 410 may include a major width 419 and a minor width 418. Major width 419 may be from 10 millimeters to 30 millimeters. Minor width 418 may be from 6 millimeters to 20 millimeters. Stem 420 may extend from the center of the contact surface 414. The various components and dimensions of drug-device combination 400, such as cap height 418, stem width 429, stem height 428, stem 420, and gripping feature 426 of drug-device combination 400 may be the same or similar to the cap width 119, cap height 118, stem width 129, and stem height 128 as described in conjunction with drug-device combination 100.

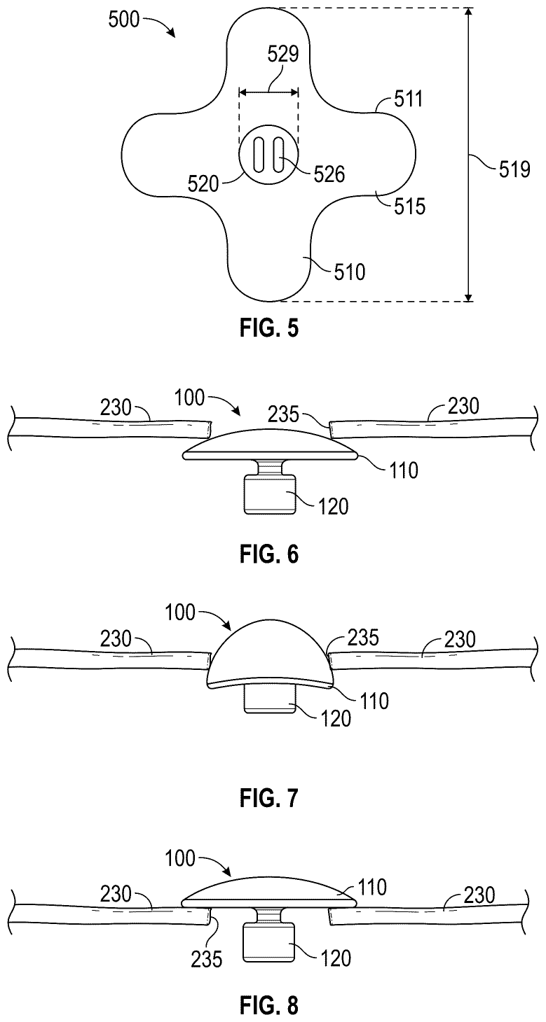

FIG. 5 is a bottom view of an alternate embodiment of the devices 100 of FIGS. 1, 300 of FIG. 3, and 400 of FIG. 4. drug-device combination 500 includes multiple legs 515, where each leg 515 can be symmetrical to the other legs. The outer perimeter 511 may be defined by the shape of each leg 515.

Device 500 may include rotational symmetry. In the embodiment shown in FIG. 5, drug-device combination 500 includes four rotationally symmetric legs 515. Each leg 515 may be a rounded solid with a flat surface on the bottom. Each leg 515 may converge with the other legs 515 at an apex forming a curved upper surface. In the embodiment illustrated, outer perimeter 511 includes the four legs 515 with each leg 515 including an arc at the end and the transition between adjacent legs 515 is also an arc.

The cap width 519 of drug-device combination 500 may be from the edge of a first leg 515 to the edge of a second leg 515 opposite the first leg 515. The cap width 519 may be from 10 millimeters to 30 millimeters. The various components and dimensions of drug-device combination 500, such as cap height 518, stem width 529, stem height 528, stem 520, and gripping feature 526 of drug-device combination 500 may be the same or similar to the cap width 119, cap height 118, stem width 129, and stem height 128 as described in conjunction with drug-device combination 100.

Any of the embodiments of the device, such as devices 100, 300, 400, and 500 as disclosed herein, herein after referred to as the device, and its various components, can be made out of a biocompatible material. The material may be a compressible and deformable material, such as an elastic material, that will regain its shape after being inserted into the sella turcica. The drug-device combination may be a non-resorbable material such as open or closed cell foam, materials with suitable durometers such as silicone or elastomeric materials. The drug-device combination may also be made from synthetic foams, polymers, plastics, or other medical grade materials. In some embodiments, the material of the drug-device combination includes a hardness/durometer of Shore 20 A or softer, i.e. a hardness up to Shore 20 A. In other embodiments, the drug-device combination includes a hardness from Shore 5 A to Shore 20 A. In yet another embodiment, the drug-device combination includes a hardness between Shore 10 A and Shore 20 A.

In particular embodiments, the material for use in the invention drug-devices for eluting the therapeutic drugs includes a copolymer of ethylene and vinyl acetate corresponding to ethylene vinyl acetate (EVA; CAS Number 24937-78-8), also referred to as poly(ethylene-vinyl acetate) (PEVA), and the like. Other drug eluting polymer materials for use herein are non-biodegradable, non-resorbable; which can readily be subsequently removed if desired. The drug-device combination may include a second material that acts as a marker for locating the drug-device combination through imaging, such as computerized tomography, magnetic resonance, and the like.

In particular embodiments, the invention drug-device combination is capable of eluting a therapeutically active drug for at least: 6 months, 9 months, 12 months, 15 months, 18 months, 21months, 24 months, or longer.

The invention drug-device combination prevents or reduces the risk of the active ingredient drug leaking into the cerebrospinal fluid or the blood system, which prevents or reduces the systemic side-effects otherwise present with current therapeutic methods using these drugs.

In particular embodiments, the invention drug-device combinations provided herein comprise one or more of the drugs for use in pituitary target therapy (e.g., pituitary gland related diseases). For example, drugs for use in pituitary target therapy include somatostatin receptor ligands (SRL), dopamine agonists, Gonadotropin Releasing Hormone (GnRH) agonists and antagonists; and the like. Exemplary somatostatin receptor ligands for use in the invention drug-device combinations are selected from: pasireotide, octreotide, lanreotide, paltusotine, and/or the like. An exemplary dopamine agonist for use in the invention drug-device combinations is cabergoline, or the like. Exemplary (GnRH) agonists for use in the invention drug-device combinations are selected from: leuprolide, goserelein, triptorelin, and/or the like. Exemplary (GnRH) antagonists for use in the invention drug-device combinations are selected from: cetrorelix, elagolix, relugolix, and/or the like.

Accordingly, provided herein are methods of treating a disease by delivering a drug substance directly to a pituitary gland, said method comprising: forming a hole in a sphenoid bone that acts as a barrier between a sella turcica and a sphenoid sinus; and inserting the invention drug-device combination provided herein. In certain embodiments, the drug substance is selected from a somatostatin receptor ligands (SRL), dopamine agonists, Gonadotropin Releasing Hormone (GnRH) agonists and/or antagonists. In particular embodiments, the drug substance is selected from the group consisting of: pasireotide, octreotide, lanreotide, paltusotine, cabergoline, leuprolide, goserelein, triptorelin, cetrorelix, elagolix, and/or relugolix. In particular embodiments, the disease is selected from: acromegaly, Cushing's disease, thyrotrophinomas, diarrhea, gastroenteropancreatic neuroendocrine tumors (GEP-NETs), carcinoid syndrome, hyperprolactinemia, Parkinsonian Syndrome, prostate cancer and/or breast cancer, and the like.

In certain embodiments, the drug-device combination is inserted by: gripping the drug-device combination of claim 1 using a surgical tool; introducing the drug-device combination into the sphenoid sinus; passing the drug-device combination through a hole in the sphenoid bone by deforming the cap; and locating the drug-device combination with its original shape such that the cap is inside the sella turcica and a contact surface of the cap is in contact with the sphenoid bone about the hole.

The drug-device combination may be configured to allow for pressure relief, such as intracranial pressure relief. The drug-device combination may also be configured to allow for injection of materials, such as a foam or gel, there through and into the sella turcica.

FIGS. 6-8 illustrate drug-device combination 100 passing through hole 235 in the sphenoid bone 230. FIG. 6 illustrates drug-device combination 100 as the tip of cap 110 initially contacts the sphenoid bone 230 at hole 235 prior to insertion of drug-device combination 100 through hole 235. Prior to exerting force to pass drug-device combination 100 through hole 235, drug-device combination 100 maintains its original shape. FIG. 7 illustrates drug-device combination 100 as it passes through hole 235. Hole 235 is smaller than the cap width 119. As drug-device combination 100 passes through hole 235, cap 110 may compress, deform, and deflect inward and downward (toward stem 120). The cap width 119 may be temporarily smaller than the diameter of hole 235 as drug-device combination 100 passes through hole 235.

FIG. 8 illustrates drug-device combination 100 after drug-device combination 100 passes through hole 235. After passing through hole 235, such as a defect in the sphenoid bone 230, drug-device combination 100 may reshape, reform, and spring back into the same or a similar shape drug-device combination 100 held prior to the insertion of drug-device combination 100 through hole 235. The various embodiments disclosed herein, including devices 300, 400, and 500 may pass through hole 235 in the same or a similar manner as drug-device combination 100. Any of the embodiments disclosed herein, may also be used for and passed through other skull based defects in different anatomic areas.

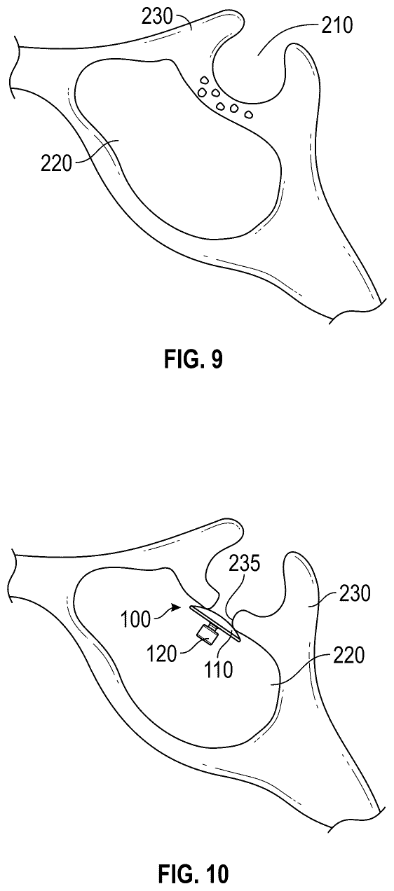

FIGS. 9-12 illustrate the insertion of drug-device combination 100 into the sella turcica 210. As illustrated in FIG. 9, prior to surgery, the sella turcica 210 is separated from the sphenoid sinus 220 by the sphenoid bone 230. During surgeries such as endoscopic transphenoidal pituitary tumor removal a hole 235 is formed in the sphenoid bone 230 at the floor of the sella turcica 210. After the surgery is completed, the drug-device combination 100 is located in an inlay position in the sella turcica 210 as illustrated in FIG. 12.

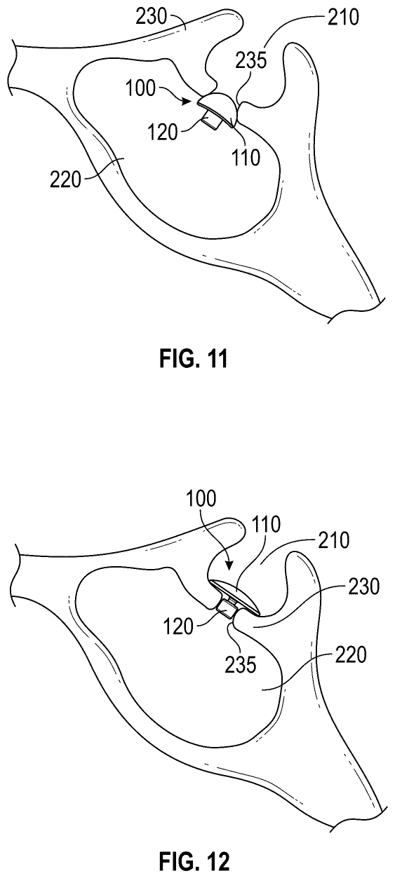

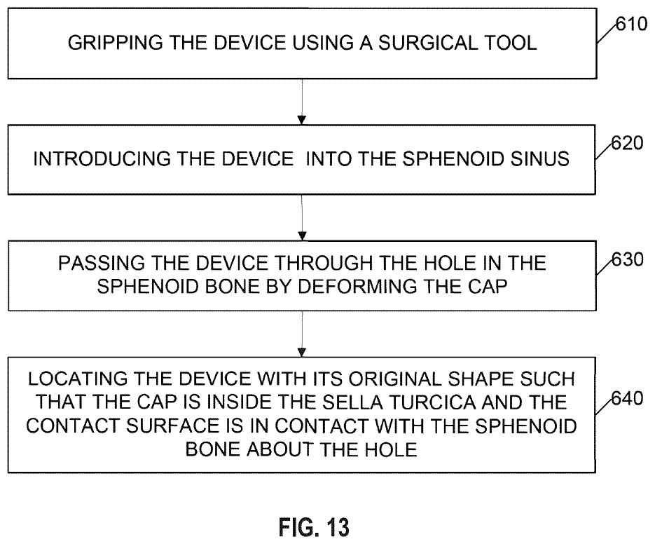

FIG. 13 is a flowchart of a method for placing (or locating) the drug-device combination 100 in an inlay position in the sella turcica 210. The method includes gripping the drug-device combination 100 using a surgical tool at step 610. The gripping feature 126 may be used to grip the drug-device combination with the surgical tool. Step 610 is followed by introducing the drug-device combination 100 into the sphenoid sinus 220 as illustrated in FIG. 10 at step 620. Step 620 is followed by passing the drug-device combination 100 through the hole 235 in the sphenoid bone 230 by deforming the cap 110 as illustrated in FIG. 11 at step 630. As the leading point or tip of cap 110 is placed within hole 235, the outer perimeter 111 of cap 110 may deflect. The spherical, ellipsoidal, or conical shape of cap 110 may lead drug-device combination 110 through hole 235 without the need for awkward maneuvers of placing a rigid bony or cartilaginous implant. Step 630 is followed by locating the drug-device combination 100 with its original shape such that cap 110 is inside the sella turcica 210 and contact surface 112 is in contact with the sphenoid bone 230 about the hole 235 as illustrated in FIG. 12 at step 640. After passing through hole 235 the outer perimeter 111 deploys above and adjacent the edge of hole 235. This may result in the inner portion of contact surface 112 lying above hole 235. drug-device combination 100 may be held into place within the sella turcica 210 by the spinal fluid pressure and by gravity. The various embodiments of the drug-device combination disclosed herein including devices 300, 400, and 500 may be located in an inlay position in the sella turcica 210 in the same or a similar method as drug-device combination 100. The various embodiments of the drug-device combination disclosed herein may be similarly located in other anatomical areas of the skull to plug or cover a skull based defect in those anatomical areas.

The compression, deformation, or deflection of the drug-device combination may give the placement of the drug-device combination within the sella turcica 210 the ease of placing an overlay graft. The elastic properties of the drug-device combination providing for the reformation of the drug-device combination into or near its original shape upon final placement may provide the structural support of an inlay graft. The drug-device combination may widen gradually to its maximal width, allowing additional mass to be added at the cap, which may provide positional stability within the sella turcica 210.

The stem of the drug-device combination may be used for placement and retrieval of the drug-device combination and may also be used for providing an element of overlay support by itself or in conjunction with a small amount of adjunctive material such as foam or surgical glue. The gripping feature may facilitate the interlocking of surgical instruments, such as Blakesly forceps during placement and retrieval of the device.

FIG. 14 illustrates the insertion of the drug-eluting drug-device combination microparticles within the sella turcica. Drug or drug-eluting compositions comprising microparticles, such as microspheres (beads) 1410, elongated microparticles, and the like, are advanced 1430 within a catheter 1420 into the sella turcica (FIG. 14). Within the catheter (or needle), a stylet, plunger, pusher wire, or the like, is used as an advancer 1430 to advance drug-eluting compositions into sell turcica. The drug-eluting compositions can be bio-durable or bio-absorbable (e.g., bio-degradable), as set forth herein. Once the drug or drug-eluting composition is administered into the sella turcica, the catheter is removed and the surgical site is closed.

In one aspect, a small bony opening or hole 235 is created in the sella turcica (similar to approach described herein in FIGS. 9-12). In one embodiment, the dura remains non-violated. In another embodiment, the dura is opened if needed. An introducer-device (also referred to herein as “introducer”) 1420 and 1520, such as a catheter, needle, or similar device, is introduced endoscopically and positioned adjacent to the pituitary gland. In particular embodiments, the introducer contains image guidance. Via the introducer, such as a catheter or the like, small drug-device delivery microparticles 1410, such as microspheres or the biocompatible polymeric particles described herein 1410, or the like, are inserted into and adjacent to the pituitary gland within the pituitary fossa. Once delivered, the catheter or needle will be withdrawn; and the surgical site will be treated with an appropriate biologic dressing for closure, such that the drug-device microparticles are confined within the pituitary fossa.

An advantage of this aspect is that there will be no F left within the sphenoid sinus, thereby reducing bioincompatibility issues. Another advantage of this aspect, is that the area can be reaccessed in a similar fashion to apply additional drug or drug-eluting microparticles, e.g., microspheres or nanospheres, and the like.

In one aspect, the therapeutic target for the invention pituitary-fossa-confined drug-device therapy is the pituitary gland for the treatment of pituitary neoplasia, hormone disorders, and the like. In this aspect, the microparticles (e.g., microspheres, and the like) will remain within the pituitary fossa and do not migrate. In their confined location within the pituitary fossa, the drug-device microparticles will elute drug and treat said pituitary disorders. In other aspects, additional targets for the invention drug-device microparticle therapy contemplated herein include the deeper CNS tissues, such as the midbrain for treating neurodegenerative procedures, infection, neoplasms, or other CNS diseases.

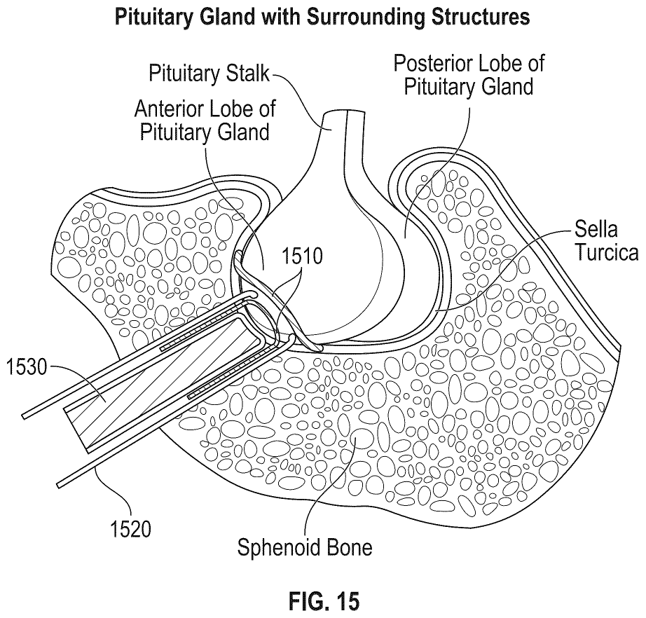

FIG. 15 illustrates the insertion of the drug-eluting drug-device combination implant (microimplant) in the shape of an elongated rod 1510 within the sella turcica. A drug-eluting composition in the form of an elongated rod implant (e.g., a microimplant) 1510 is pre-loaded in an introducer 1520 (e.g., a catheter or needle, or the like). Within the catheter (or needle), a stylet, plunger, pusher wire, or the like, is used as an advancer 1530 to advance drug-eluting compositions into sell turcica. After advancement of the microimplant into the sella turcica through the introducer, the implant reforms to its original shape, i.e., the implants demonstrates shape memory. The introducer (catheter 1520) is then removed and the surgical site closed with biologic dressing. The drug-eluting compositions can be bio-durable or bio-absorbable (e.g., bio-degradable), as set forth herein.

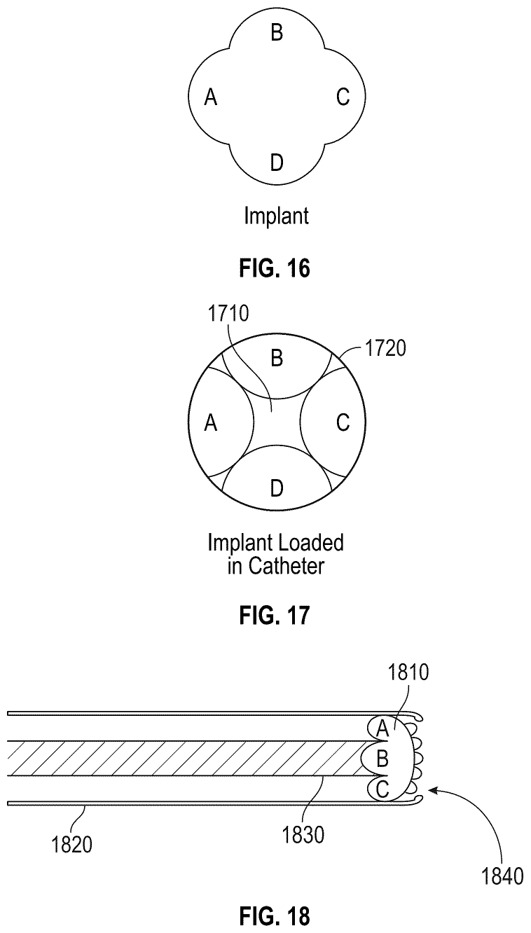

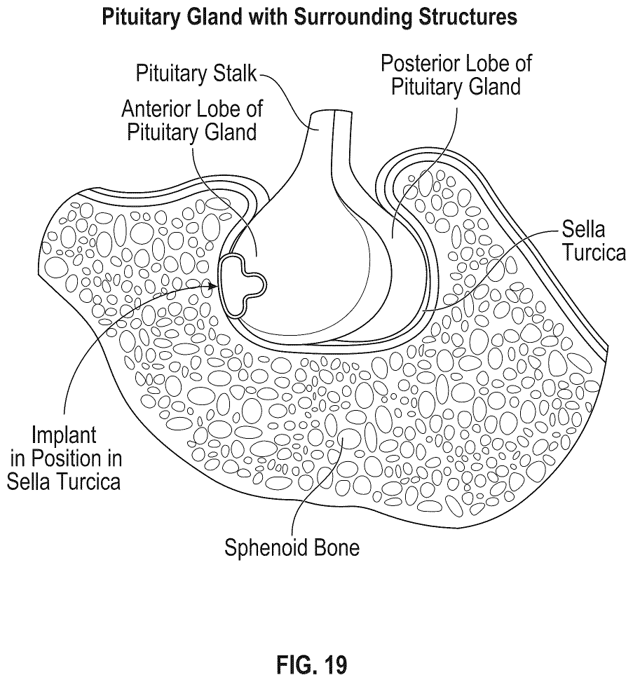

In this embodiment, the implant can be any shape so long as it is capable of deforming during the loading phase via the catheter and reforming to its original shape once inserted into the sella turcica or the pituitary fossa. Suitable shapes for use in the implants inserted within the sella turcica or pituitary fossa (as depicted in FIGS. 15-19) include clover (FIG. 16), crescent, star-like, sphere, elongated rod (FIG. 15; 1510), and the like shapes. In certain embodiments, the implant further comprises nodules 1840, ridges and/or small bumps on the leading edge and/or on the side touching the gland for stability (see FIG. 18; 1840). In a particular embodiment, the use of a “clover” shape implant (FIG. 16-19) facilitates the ease of catheter loading and unloading, where the flanges (e.g., A-D) of the clover deflect upon loading and return to shape upon delivery.

In other embodiments, the implants inserted into the sella turcica do not reform to their original shape after insertion, and still maintain the ability to elute the drug into the sella turcica and/or the pituitary fossa.

In one aspect, a small bony opening or hole 235 is created in the sella turcica (similar to approach described herein in FIGS. 9-12). In one embodiment, the dura remains non-violated. In another embodiment, the dura is opened if needed. An introducer-device (also referred to herein as “introducer”), such as a catheter or needle or similar device, is introduced endoscopically and positioned adjacent to the pituitary gland. In particular embodiments, the introducer contains image guidance to facilitate the drug-device delivery. Via the introducer, a small drug-device delivery implant (microimplant) is inserted into and adjacent to the pituitary gland within the pituitary fossa. Once delivered, the needle will be withdrawn; and the surgical site will be treated with an appropriate biologic dressing for closure, such that the drug-device implant(s) are confined within the pituitary fossa.

An advantage of this aspect is that there will be no implant left within the sphenoid sinus, thereby reducing bioincompatibility issues. Another advantage of this aspect, is that the area can be reaccessed in a similar fashion to apply additional drug or drug-eluting implants, and the like.

In one aspect, the therapeutic target for the invention pituitary-fossa-confined drug-device microimplant therapy is the pituitary gland for the treatment of pituitary neoplasia, hormone disorders, and the like. In this aspect, the implant remains within the pituitary fossa and does not migrate. In their confined location within the pituitary fossa, the drug-device implants will elute drug and treat said pituitary disorders. In other aspects, additional targets for the invention drug-device microimplant therapy contemplated herein include the deeper CNS tissues, such as the midbrain for treating neurodegenerative procedures, infection, neoplasms, or other CNS diseases.

Also contemplated herein is the co-insertion of the drug-device implants and microparticles into pituitary fossa in combination. In this aspect, drug or drug-eluting compositions including microimplants and microparticles, such as microspheres (beads), elongated microparticles, and the like, are advanced via a catheter and/or introducer (FIG. 18) into the sella turcica (FIGS. 14, 15, 18 and 19). In this aspect, the implants and microparticles (e.g., microspheres, and the like) will remain within the pituitary fossa and do not migrate. In their confined location within the pituitary fossa, the drug-device implants and/or microparticles will elute drug and treat said pituitary disorders.

Suitable drug-eluting compositions for use in the invention methods and drug-device combinations include polymeric compositions that have been used in forming various implantable medical devices and injectable drug formulations for sustained and controlled local delivery of therapeutic agents (i.e., drugs). These drug-containing polymeric compositions are typically formed by dissolving one or more therapeutic agent and one or more biocompatible polymers in one or more solvents, followed by removing the solvents to form a solidified drug-containing polymeric composition. The solvent removal or solidification can be carried out using various techniques, including, but not limited to: spray drying (for preparation of coatings), solvent casting or spin coating (for preparation of thin films or membranes), and spinning (for preparation of fibers).

In particular embodiments, the solidified drug-containing polymeric compositions so formed contain the therapeutic agents in an amorphous phase. See, e.g., U.S. Pat. No. 4,389,330; U.S. Pat. No. 4,530,840; U.S. Pat. No. 5,688,801; U.S. Pat. No. 6,803,055; U.S. Pat. No. 10,195,138; and the like; each of which are incorporated by reference in their entirety for all purposes. In other embodiments, the drug-containing polymeric compositions for use herein contain the therapeutic agents (drugs), or at least a portion thereof, in a more stable crystalline phase (see, e.g., U.S. Pat. No. 7,842,312; and the like; each of which are incorporated by reference in their entirety for all purposes).

In certain aspects, the drug-containing polymeric compositions contain little or no amorphous therapeutic agents, i.e., a major portion (i.e., >50%) of the therapeutic agents contained in such compositions are in the stable crystalline phase. In another aspect, the drug-containing polymeric compositions each comprises at least one therapeutic agent encapsulated in at least one biocompatible polymer, while more than 75% of the therapeutic agent in the composition is crystalline. In another aspect, more than 90% or more than 95% of the therapeutic agent in the composition is crystalline. In one embodiment, the composition is essentially free of amorphous therapeutic agent.

In certain aspects, the at least one therapeutic agent as described hereinabove is encapsulated into at least one biocompatible polymer, which provides structural support for the therapeutic agent, functions as a carrier matrix therefore, and controls the release thereof. The at least one biocompatible polymer may be any suitable biocompatible polymer or any suitable mixture of polymers, including, but not limited to: biocompatible addition polymers and biocompatible condensation polymers. Further, the at least one biocompatible polymer of the present invention may either be biostable or biodegradable, and it may even comprise a polymer blends of a biostable polymer and a biodegradable polymer.

Biostable polymers that are suitable for use in this invention include, but are not limited to: polyurethane, silicones, polyesters, polyolefins, polyamides, poly(esteramide), polycaprolactam, polyimide, polyvinyl chloride, polyvinyl methyl ether, polyvinyl alcohol, acrylic polymers and copolymers, polyacrylonitrile; polystyrene copolymers of vinyl monomers with olefins (such as styrene acrylonitrile copolymers, ethylene methyl methacrylate copolymers, ethylene vinyl acetate), polyethers, rayons, cellulosics (such as cellulose acetate, cellulose nitrate, cellulose propionate, etc.), parylene and derivatives thereof; and mixtures and copolymers of the foregoing.

Biodegradable polymers that can be used in this invention include, but are not limited to: polylactic acid (PLA), polyglycolic acid (PGA), copolymers of lactic acid and glycolic acid (PLGA), polycaprolactone, polyphosphoester, polyorthoester, poly(hydroxy butyrate), poly(dioxanone), poly(hydroxy valerate), poly(hydroxy butyrate-co-valerate), poly(glycolide-co-trimethylene carbonate), polyanhydrides, poly(ester-amide), polyphosphazene, poly(phosphoester-urethane), poly(amino acids), biopolymeric molecules such as fibrin, fibrinogen, cellulose, starch, collagen and hyaluronic acid, and mixtures and copolymers of the foregoing.

In particular embodiments, the at least one biocompatible polymer is a biodegradable polymer selected from the group consisting of PLA, PGA, PLGA, and mixtures thereof. More preferably, the polymeric material used by the present invention comprises the PLGA copolymer. The PLA, PGA, or PLGA polymers may be any of D-, L- and D-/L-configuration.

The at least one biocompatible polymer may form a substantially continuous polymeric matrix with the at least one therapeutic agent encapsulated therein. The substantially continuous polymeric matrix can either constitute at least a portion of an implantable medical device or form a coating over at least a portion of the implantable medical device. Various implantable medical devices can be formed or coated by the drug-containing polymeric composition to effectuate controlled local drug delivery. For example, such implantable medical devices may be selected from clovers (FIG. 16), crescents, star-like, spheres, elongated rods (FIG. 15; 1510), grafts, patches, catheters, guide wires, balloons, filters, and the like.

Alternatively, the at least one biocompatible polymer may form polymeric particles (e.g., microparticles or nanoparticles; FIG. 14; 1410) with the at least one therapeutic agent encapsulated therein. The polymeric particles may have any suitable sizes (e.g., from about 1 nm to about 1 mm in average diameter) and shapes (e.g., sphere, ellipsoid, etc.). In particular embodiments, the at least one biocompatible polymer forms nano-and/or micro-particles that are suitable for injection via the introducer. The term “nano-particles” or “micro-particles” is used throughout the present invention to denote carrier structures (FIG. 14; 1410 that are biocompatible and have sufficient resistance to chemical and/or physical destruction by the environment of use such that a sufficient amount of the nano-particles and/or micro-particles remain substantially intact after injection into a target site in the arterial wall. Typically, the nano-particles have sizes ranging from about 1 nm to about 1000 nm, with sizes from about 100 nm to about 500 nm being more preferred. The micro-particles have sizes ranging from about 1 μm to about 1000 μm, with sizes from about 10 μm to about 200 μm being more preferred. The pharmacologically active agent as described hereinabove is loaded within and/or on the surfaces of the nano-particles and/or micro-particles.

In a particular embodiment, the at least one therapeutic agent are first formed into crystalline particles of desired sizes, which are then encapsulated into the at least one biocompatible polymer. In certain embodiments, the crystalline particles of the therapeutic agent have an average particle size ranging from about 50 nm to about 50 μm, and typically from about 100 nm to about 200 nm.

Nanotechnology provides new and enhanced particle formulation processes and offers a wide range of options for achieving drug particles in the micro-and nano-size range. Some of the new developments in nanotechnology have successfully achieved particle engineering by using molecular scaffolds like dendrimers (polyvalent molecules) and fullerenes (i.e., C-60 “bucky balls”). The small-size drug particles that can be formed by using nanotechnology are particularly useful for formulating poorly soluble drugs, since the reduced drug particle sizes significantly improve the bioavailability of such drugs, by providing higher surface area and accelerating dissolution and absorption of such drugs by the body.

Further, conventional techniques, such as milling (either dry or wet), supercritical extraction, spray drying, precipitation, and recrystallization, can also be used to prepare micro-and nano-size drug particles. Milling is a well-established micronization technique for obtaining desired micro-and nano-size drug particles (either dry or suspended in liquid) with well controlled size distribution.

If the particle sizes of the crystalline drug particles as provided are already suitable for forming a polymeric composition that can be subsequently used to form or coat a drug-eluting implantable medical device or drug-eluting micro-and/or nano-particles, then such crystalline drug particles can be directly used for forming the polymeric composition. However, if the particle sizes of the crystalline drug particles as provided are too large, the above-described methods can be readily used, either separately or in combination, to reduce the particles size down to a desired size range.

In a particular embodiment, the crystalline particles are encapsulated into the at least one biocompatible polymer by a process that uses a polymeric solution. Specifically, the polymeric solution comprises the at least one biocompatible polymer as dissolved in a solvent system, which may comprise a single solvent or multiple solvents, provided that the crystalline particles of the at least one therapeutic agent are insoluble in such a solvent system. In this manner, the crystalline particles can retain their crystallinity even after mixing with the polymeric solution, and the mixture can then be processed, i.e., to remove all or substantially all of the solvent(s), to form the drug-containing polymeric composition with the crystalline particles of therapeutic agent encapsulated therein.

The drug/polymeric solution mixture can be either formed into or coated over at least a portion of an implantable medical device before the solvent removal. In this manner, a substantially continuous biocompatible matrix is formed after the solvent removal, which constitutes at least a portion of the implantable medical device, or a coating over such an implantable medical device, with the crystalline particles of the therapeutic agent encapsulated therein. As set forth herein, suitable shapes for use in the implants inserted within the sella turcica or pituitary fossa (as depicted in FIGS. 15-19) include clover (FIG. 16), crescent, star-like, sphere, elongated rod (FIG. 15; 1510), and the like shapes.

Alternatively, the crystalline drug particles are first encapsulated individually by a protective coating layer that is not dissolvable in the polymeric solution, before mixed with the polymeric solution. In this manner, the crystalline drug particles, being individually encapsulated and protected by the protective material layer, will retain their crystallinity in the polymeric solution, regardless of whether the drug particle itself is soluble or insoluble in the polymeric solution. In other words, the protective material layer forms a barrier for the drug particles to prevent the drug particles from being dissolved by the solvent(s) contained in the polymeric solution, thereby preserving the crystalline morphology of the drug particles.

Micro-encapsulation is a process in which tiny particles or droplets are individually encapsulated by protective coating layers to form small capsules with many useful properties. The material inside the microcapsule is usually known as the core, which is surrounded by a wall, sometimes referred to as a shell, coating, or membrane. Most of the microcapsules have diameters between a few micrometers and a few millimeters. The core of a microcapsule may be a single crystal, a jagged particle, an emulsion, a suspension of solids, or a suspension of smaller microcapsules.

There are several reasons for preparing micro-encapsulations. In some cases, the core must be isolated from its environment, as in isolating an active ingredient from the deteriorating effects of oxygen, retarding evaporation of a volatile core, improving the handling and flow properties of a sticky material, or isolating a reactive core from chemical attack. In other cases, the objective is not to isolate the core completely but to control the rate at which it leaves the microcapsule, as in the controlled release of drugs.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope fully encompasses other embodiments that may become obvious to those skilled in the art.

We claim:

1. A drug-device combination for direct delivery of a drug to a pituitary gland, the drug-device comprising:
a cap formed of a deformable material, the cap including
a contact surface forming a base of the cap, the contact surface including an outer perimeter, and
an upper surface including an apex offset from the contact surface at a cap height, the upper surface extending from the apex to the outer perimeter;
a stem extending from the contact surface in a direction opposite the apex, wherein the drug-device combination covers a hole formed in a sphenoid bone between a sella turcica and a sphenoid sinus; and
a drug substance that is therapeutically effective for a pituitary gland disorder.

2. The drug-device combination of claim 1, wherein the drug substance is selected from a somatostatin receptor ligand (SRL) or a dopamine agonist.

3. The drug-device combination of claim 1, wherein the drug substance is selected from the group consisting of: pasireotide, octreotide, lanreotide, paltusotine, cabergoline, leuprolide, goserelein, triptorelin, cetrorelix, elagolix, and/or relugolix.

4. A method for locating a drug-device combination in an inlay position in the sella turcica, the drug-device combination including a cap formed of deformable material and a stem extending from the cap, the method comprising:
gripping the drug-device combination of claim 1 using a surgical tool;
introducing the drug-device combination into the sphenoid sinus;
passing the drug-device combination through a hole in the sphenoid bone by deforming the cap; and
locating the drug-device combination with its original shape such that the cap is inside the sella turcica and a contact surface of the cap is in contact with the sphenoid bone about the hole.

5. The method of claim 4, wherein gripping the drug-device combination using a surgical tool includes gripping a ridge of a gripping feature located at an end of the stem distal to the cap.

6. A method of treating a disease by delivering a drug substance directly to a pituitary gland, said method comprising: forming a hole in a sphenoid bone that acts as a barrier between a sella turcica and a sphenoid sinus; and inserting a drug-device combination implant.

7. The method of claim 6, wherein the drug-device combination implant is inserted by using a catheter, needle or gripping the drug-device combination using a surgical tool; introducing the drug-device combination implant into the sphenoid sinus; passing the drug-device combination implant through a hole in the sphenoid bone by deforming the drug-device combination implant; and locating the drug-device combination implant such that the drug-device combination implant is inside the sella turcica and in contact with the sphenoid bone about the hole.

8. The method of claim 6, wherein the drug-device combination implant regains its original shape.

9. The method of claim 6, wherein the drug substance is selected from a somatostatin receptor ligand (SRL) or a dopamine agonist.

10. The method of claim 6, wherein the drug substance is selected from the group consisting of:
pasireotide, octreotide, lanreotide, paltusotine, cabergoline, leuprolide, goserelein, triptorelin, cetrorelix, elagolix, and/or relugolix.

11. The method of claim 6 wherein the disease is selected from: acromegaly, Cushing's disease, thyrotrophinomas, diarrhea, gastroenteropancreatic neuroendocrine tumors (GEP-NETs), carcinoid syndrome, hyperprolactinemia, Parkinsonian Syndrome, prostate cancer and/or breast cancer.

12. The method of claim 6, wherein the drug-device combination implant is configured to elute drug substance into the sella turcica for direct contact with the pituitary gland, wherein the drug substance is effectively confined within the sella turcica and prevented from contact with the CSF or blood system.

13. A method for placing a drug-device combination within a pituitary fossa, the method comprising:
forming a hole in a sphenoid bone that acts as a barrier between a sella turcica and a sphenoid sinus using a surgical tool;
introducing the drug-device combination into the pituitary fossa via an introducer inserted into the hole;
withdrawing the introducer; and
surgically closing the hole,
wherein the drug-device combination comprises a drug substance and an implant and/or microparticle.

14. The method of claim 13, wherein the introducer is a needle or catheter.

15. The method of claim 13, wherein the drug substance is selected from somatostatin receptor ligands (SRL), dopamine agonists, Gonadotropin Releasing Hormone (GnRH) agonists and/or GnRH antagonists.

16. The method of claim 13, wherein the drug substance is selected from the group consisting of: pasireotide, octreotide, lanreotide, paltusotine, cabergoline, leuprolide, goserelein, triptorelin, cetrorelix, elagolix, and/or relugolix.

17. The method of claim 13, wherein the drug-device combination implant is configured to elute drug substance into the sella turcica for direct contact with the pituitary gland, wherein the drug substance is effectively confined within the sella turcica and prevented from contact with the CSF or blood system.

18. A drug-device combination for direct delivery of a drug to a pituitary gland, the drug-device comprising:
an implant formed of a deformable material; and
a drug substance that is therapeutically effective for a pituitary gland disorder.

19. The drug-device combination of claim 18, wherein the drug substance is selected from a somatostatin receptor ligand (SRL) or a dopamine agonist; or is selected from the group consisting of: pasireotide, octreotide, lanreotide, paltusotine, cabergoline, leuprolide, goserelein, triptorelin, cetrorelix, elagolix, and/or relugolix.

20. The drug-device combination of claim 18, wherein the deformable material is an elastic material; or wherein the deformable material is ethylene vinyl acetate (EVA); or wherein the implant comprises a microparticle, microspheres and/or nanosphere.
